(19) United States
(12) Patent Application Publication
(10) Pub. No.: US 2018/0275115 A1
(43) Pub. Date: Sep. 27, 2018

(54) METHOD OF IDENTIFYING AND TREATING CARDIOVASCULAR DISEASE AND PRE-CARDIOVASCULAR DISEASE STATE

(71) Applicant: Board of Supervisors of Louisiana State University and Agricultural and Mechanical College, Baton Rouge, LA (US)

(72) Inventors: Christopher KEVIL, Shreveport, LA (US); Matthew DESHOTELS, Shreveport, LA (US)

(21) Appl. No.: 15/935,557

(22) Filed: Mar. 26, 2018

Related U.S. Application Data
(60) Provisional application No. 62/476,344, filed on Mar. 24, 2017.

(51) Int. Cl. G01N 33/487; G01N 30/86

(52) U.S. Cl. CPC G01N 33/487; G01N 2030/027; G01N 2800/32; G01N 30/8675

(57) ABSTRACT

The disclosed invention is directed to devices, therapeutics and methods of diagnosing and treating cardio vascular disease in a patient comprising measuring plasma levels of one of free sulfide, acid labile sulfide (ALS), bound sulfane sulfur (BSS), total sulfide metabolites, and some combination thereof, diagnosing, based on plasma levels, the patient with cardio vascular disease, and administering a therapeutically effective dose of a pharmaceutical composition to the patient. According to a further embodiment, the patient is diagnosed with cardio vascular disease if the patient has a polymorphism in a cystathionine gamma-lyase gene but not a cystathionine-beta-synthase gene. According to a further embodiment, the patient is diagnosed with peripheral atrial disease if the patient is African American and the plasma BSS level is significantly lower than an average control plasma BSS level. According to a further embodiment, the patient is diagnosed with cardio vascular disease if the patient has a one of lower BSS plasma level and lower total sulfide plasma level than a respective average control BSS plasma level and total sulfide plasma level. According to a further embodiment, the patient is diagnosed with cardio vascular disease if the patient has a both lower BSS plasma level and lower total sulfide plasma level than a respective average control BSS plasma level and total sulfide plasma level. According to a further embodiment, the patient is diagnosed with cardiac arterial disease and peripheral arterial disease if the patient possesses a CTH 1364 G>T Single Nucleotide Polymorphism. According to a further embodiment, the patient is diagnosed with cardiac arterial disease if the patient possesses an eNOS 894 G>T Single Nucleotide Polymorphism.

### CROSS REFERENCE TO RELATED APPLICATIONS/PRIORITY

The present invention claims priority to United States Provisional Patent Application No. 62/476,344 filed Mar. 24, 2017, which is incorporated by reference into the present disclosure as if fully restated herein. Any conflict between the incorporated material and the specific teachings of this disclosure shall be resolved in favor of the latter. Likewise, any conflict between an art-understood definition of a word or phrase and a definition of the word or phrase as specifically taught in this disclosure shall be resolved in favor of the latter.

### STATEMENT REGARDING FEDERALLY SPONSORED RESEARCH OR DEVELOPMENT

This invention was made with government support under Grant No. HL113303 awarded by the National Institutes of Health. The government has certain rights in the invention.

### BACKGROUND

Hydrogen sulfide (H2S) is an important physiological and pathophysiological signaling molecule in the cardiovascular system influencing vascular tone, cytoprotective responses, redox reactions, vascular adaptation, and mitochondrial respiration. However, bioavailable levels of H2S in its various biochemical metabolite forms during clinical cardiovascular disease remain poorly understood by the current art.

### SUMMARY

Wherefore, it is an object of the present invention to overcome the above mentioned shortcomings and drawbacks associated with the current technology.

The disclosed invention is directed to devices, therapeutics, and methods of diagnosing and treating cardio vascular disease in a patient comprising measuring plasma levels of one of free sulfide, acid labile sulfide (ALS), bound sulfane sulfur (BSS), total sulfide metabolites, and some combination thereof, diagnosing, based on plasma levels, the patient with cardio vascular disease, and administering a therapeutically effective dose of a pharmaceutical composition to the patient. According to a further embodiment, the patient is diagnosed with cardio vascular disease if the patient has a polymorphism in a cystathionine gamma-lyase gene but not a cystathionine-beta-synthase gene. According to a further embodiment, the patient is diagnosed with peripheral atrial disease if the patient is African American and the plasma BSS level is significantly lower than an average control plasma BSS level. According to a further embodiment, the patient is diagnosed with cardio vascular disease if the patient has a one of lower BSS plasma level and lower total sulfide plasma level than a respective average control BSS plasma level and total sulfide plasma level. According to a further embodiment, the patient is diagnosed with cardio vascular disease if the patient has a both lower BSS plasma level and lower total sulfide plasma level than a respective average control BSS plasma level and total sulfide plasma level. According to a further embodiment, the patient is diagnosed with cardiac arterial disease and peripheral arterial disease if the patient possesses a CTH 1364 G>T Single Nucleotide Polymorphism. According to a further embodiment, the patient is diagnosed with cardiac arterial disease if the patient possesses an eNOS 894 G>T Single Nucleotide Polymorphisms. According to a further embodiment, the patient is diagnosed with cardiac arterial disease if the patient possesses an eNOS 894 G>T Single Nucleotide Polymorphism and a CTH 1364 G>T Single Nucleotide Polymorphism. According to a further embodiment, the patient is diagnosed with one of cardiac arterial disease and peripheral arterial disease if the patient is Caucasian and the plasma total sulfide level is lower than an average plasma total sulfide level. According to a further embodiment, the patient is diagnosed with one of cardiac arterial disease and peripheral arterial disease if the patient is Caucasian and the plasma BSS level lower than an average control plasma BSS level. According to a further embodiment, the patient is diagnosed with cardiac arterial disease if the patient is an African American and the plasma BSS level is significantly elevated compared to an average plasma BSS level of peripheral arterial disease patients. According to a further embodiment, the patient is diagnosed with cardio vascular disease if the patient is a Caucasian female and one of plasma total sulfide level and plasma ALS level is significantly lower than a respective average total sulfide level and plasma ALS level of African American female CVD patients. According to a further embodiment, the patient is diagnosed with cardio vascular disease if the patient is a Caucasian female and both plasma total sulfide level and plasma ALS level is significantly lower than a respective average total sulfide level and average plasma ALS level of African American female CVD patients. According to a further embodiment, the patient is diagnosed with cardio vascular disease if the patient is a female and one of plasma total sulfide level and plasma ALS level is significantly lower than a respective average control plasma total sulfide level and plasma ALS level. According to a further embodiment, the patient is diagnosed with cardio vascular disease if the patient is a female and both plasma total sulfide level and plasma ALS level is significantly lower than a respective average control plasma total sulfide level and plasma ALS level. According to a further embodiment, the patient is diagnosed with cardio vascular disease if the patient is Caucasian and one of plasma total sulfide level, plasma ALS level, and plasma BSS level is significantly lower than a respective average control plasma total sulfide level, plasma ALS level, and plasma BSS level. According to a further embodiment, the patient is diagnosed with cardio vascular disease if the patient is Caucasian and each of plasma total sulfide level, plasma ALS level, and plasma BSS level is significantly lower than a respective average control plasma total sulfide level, plasma ALS level, and plasma BSS level. According to a further embodiment, the patient is diagnosed with cardio vascular disease if the patient is African Americans and has one of lower plasma total sulfide level and lower plasma BSS level compared to respective average African American control plasma total sulfide level and plasma BSS level. According to a further embodiment, the patient is diagnosed with cardio vascular disease if the patient is African Americans and has both lower plasma total sulfide level and lower plasma BSS level compared to respective average African American control plasma total sulfide level and plasma BSS level. According to a further embodiment, the pharmaceutical composition is one of aspirin, a beta blocker, nitroglycerin, an angiotensin-converting enzyme (ACE) inhibitor, an angiotensin II receptor blocker (ARBs), nitrite, a hypertension medication, a medication to control blood sugar, clopidogrel, cilostazol, pentoxifylline, and a cholesterol-modifying medication, a CTH polymorphism therapeutic, an eNOS polymorphism therapeutic, and some combination thereof.

The inventors performed a case-controlled study to quantify and compare the bioavailability of various biochemical forms of H2S in patients with and without cardiovascular disease (CVD). In the study, the inventors used the reverse-phase high performance liquid chromatography monobromobimane assay to analytically measure bioavailable pools of H2S. Single nucleotide polymorphisms (SNPs) were also identified using DNA Pyrosequencing. The inventors found that plasma acid labile sulfide levels were significantly reduced in Caucasian females with CVD compared with those without the disease. Conversely, plasma bound sulfane sulfur levels were significantly reduced in Caucasian males with CVD compared with those without the disease. Surprisingly, gender differences of H2S bioavailability were not observed in African Americans, although H2S bioavailability was significantly lower overall in African Americans compared to Caucasians. The inventors also performed SNP analysis of H2S synthesizing enzymes and found a significant increase in cystathionine gamma-lyase (CTH) 1364 G-T allele frequency in patients with CVD compared to controls. Lastly, plasma H2S bioavailability was found to be predictive for cardiovascular disease in Caucasian subjects as determined by receiver operator characteristic analysis. These findings reveal that plasma H2S bioavailability could be considered a biomarker for CVD in an ethnic and gender manner. Cystathionine gamma-lyase 1346 G-T SNP might also contribute to the risk of cardiovascular disease development, and evidences that treatment of the CTH polymorphism should aid at preventing and/or treating cardiovascular disease or pre-cardiovascular disease.

In this study, the inventors disclose findings of a clinical case-control study to accurately measure the amounts of different sulfide biochemical pools, namely ALS (with free sulfide combined), BSS, and total sulfide in subjects with coronary artery disease (CAD) or peripheral artery disease (PAD) compared to those without disease (controls). By combining clinically validated diagnoses with thoroughly established analytical chemistry techniques, these data provide important new insight regarding variations in bioavailability of sulfide biochemical pools and their association with cardiovascular disease states.

The present invention relates to pharmaceutical compositions of a therapeutic (e.g., aspirin, beta blockers, nitroglycerin, angiotensin-converting enzyme (ACE) inhibitors and angiotensin II receptor blockers (ARBs), nitrite, hypertension medications, medication to control blood sugar, clopidogrel, cilostazol, pentoxifylline, and cholesterol-modifying medications, including statins, niacin, fibrates and bile acid sequestrants, a CTH polymorphism therapeutic, and an eNOS polymorphism therapeutic), or a pharmaceutically acceptable salt, solvate, ester, amide, clathrate, stereoisomer, enantiomer, prodrug or analogs thereof, and use of these compositions for the treatment of CVD, including CAD and PAD.

In some embodiments, the therapeutic, or a pharmaceutically acceptable salt, solvate, or prodrug thereof, is administered as a pharmaceutical composition that further includes a pharmaceutically acceptable excipient.

In some embodiments, administration of the pharmaceutical composition to a human results in a peak plasma concentration of the therapeutic between 0.05 μM-10 μM (e.g., between 0.05 μM-5 μM).

In some embodiments, the peak plasma concentration of the therapeutic is maintained for up to 14 hours. In other embodiments, the peak plasma concentration of the therapeutic is maintained for up to 1 hour.

In some embodiments, the condition is CVD.

In certain embodiments, the CVD is mild to moderate CVD.

In further embodiments, the CVD is moderate to severe CVD.

In other embodiments, the therapeutic is administered at a dose that is between 0.05 mg-5 mg/kg weight of the human.

In certain embodiments, the pharmaceutical composition is formulated for oral administration.

In other embodiments, the pharmaceutical composition is formulated for extended release.

In still other embodiments, the pharmaceutical composition is formulated for immediate release.

In some embodiments, the pharmaceutical composition is administered concurrently with one or more additional therapeutic agents for the treatment or prevention of CVD.

In some embodiments, the therapeutic, or a pharmaceutically acceptable salt, solvate, or prodrug thereof, is administered as a pharmaceutical composition that further includes a pharmaceutically acceptable excipient.

In some embodiments, administration of the pharmaceutical composition to a human results in a peak plasma concentration of the therapeutic between 0.05 μM-10 μM (e.g., between 0.05 μM-5 μM).

In some embodiments, the peak plasma concentration of the therapeutic is maintained for up to 14 hours. In other embodiments, the peak plasma concentration of the therapeutic is maintained for up to 1 hour.

In other embodiments, the therapeutic is administered at a dose that is between 0.05 mg-5 mg/kg weight of the human.

In certain embodiments, the pharmaceutical composition is formulated for oral administration.

In other embodiments, the pharmaceutical composition is formulated for extended release.

In still other embodiments, the pharmaceutical composition is formulated for immediate release.

As used herein, the term “delayed release” includes a pharmaceutical preparation, e.g., an orally administered formulation, which passes through the stomach substantially intact and dissolves in the small and/or large intestine (e.g., the colon). In some embodiments, delayed release of the active agent (e.g., a therapeutic as described herein) results from the use of an enteric coating of an oral medication (e.g., an oral dosage form).

The term an “effective amount” of an agent, as used herein, is that amount sufficient to effect beneficial or desired results, such as clinical results, and, as such, an “effective amount” depends upon the context in which it is being applied.

The terms “extended release” or “sustained release” interchangeably include a drug formulation that provides for gradual release of a drug over an extended period of time, e.g., 6-12 hours or more, compared to an immediate release formulation of the same drug. Preferably, although not necessarily, results in substantially constant blood levels of a drug over an extended time period that are within therapeutic levels and fall within a peak plasma concentration range that is between, for example, 0.05-10 μM, 0.1-10 μM, 0.1-5.0 μM, or 0.1-1 μM.

As used herein, the terms “formulated for enteric release” and “enteric formulation” include pharmaceutical compositions, e.g., oral dosage forms, for oral administration able to provide protection from dissolution in the high acid (low pH) environment of the stomach. Enteric formulations can be obtained by, for example, incorporating into the pharmaceutical composition a polymer resistant to dissolution in gastric juices. In some embodiments, the polymers have an optimum pH for dissolution in the range of approx. 5.0 to 7.0 (“pH sensitive polymers”). Exemplary polymers include methacrylate acid copolymers that are known by the trade name Eudragit® (e.g., Eudragit® L100, Eudragit® S100, Eudragit® L-30D, Eudragit® FS 30D, and Eudragit® L100-55), cellulose acetate phthalate, cellulose acetate trimellitiate, polyvinyl acetate phthalate (e.g., Coaterie), hydroxyethylcellulose phthalate, hydroxypropyl methylcellulose phthalate, or shellac, or an aqueous dispersion thereof. Aqueous dispersions of these polymers include dispersions of cellulose acetate phthalate (Aquateric®) or shellac (e.g., MarCoat 125 and 125N). An enteric formulation reduces the percentage of the administered dose released into the stomach by at least 50%, 60%, 70%, 80%, 90%, 95%, or even 98% in comparison to an immediate release formulation. Where such a polymer coats a tablet or capsule, this coat is also referred to as an “enteric coating.”

The term “immediate release” includes where the agent (e.g., therapeutic), as formulated in a unit dosage form, has a dissolution release profile under in vitro conditions in which at least 55%, 65%, 75%, 85%, or 95% of the agent is released within the first two hours of administration to, e.g., a human. Desirably, the agent formulated in a unit dosage has a dissolution release profile under in vitro conditions in which at least 50%, 65%, 75%, 85%, 90%, or 95% A of the agent is released within the first 30 minutes, 45 minutes, or 60 minutes of administration.

The term “pharmaceutical composition,” as used herein, includes a composition containing a compound described herein (e.g., aspirin, beta blockers, nitroglycerin, angiotensin-converting enzyme (ACE) inhibitors and angiotensin II receptor blockers (ARBs), nitrite, hypertension medications, medication to control blood sugar, clopidogrel, cilostazol, pentoxifylline, and cholesterol-modifying medications, including statins, niacin, fibrates and bile acid sequestrants, or any pharmaceutically acceptable salt, solvate, or prodrug thereof), formulated with a pharmaceutically acceptable excipient, and typically manufactured or sold with the approval of a governmental regulatory agency as part of a therapeutic regimen for the treatment of disease in a mammal.

Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other formulation described herein.

A “pharmaceutically acceptable excipient,” as used herein, includes any ingredient other than the compounds described herein (for example, a vehicle capable of suspending or dissolving the active compound) and having the properties of being nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, or waters of hydration. Exemplary excipients include, but are not limited to: butylated hydroxytoluene (BHT), calcium carbonate, calcium phosphate (dibasic), calcium stearate, croscarmellose, cross-linked polyvinyl pyrrolidone, citric acid, crospovidone, cysteine, ethylcellulose, gelatin, hydroxypropyl cellulose, hydroxypropyl methylcellulose, lactose, magnesium stearate, maltitol, maltose, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol.

The term “pharmaceutically acceptable prodrugs” as used herein, includes those prodrugs of the compounds of the present invention which are, within the scope of sound medical judgment, suitable for use in contact with the tissues of humans and animals with undue toxicity, irritation, allergic response, and the like, commensurate with a reasonable benefit/risk ratio, and effective for their intended use, as well as the zwitterionic forms, where possible, of the compounds of the invention.

The term “pharmaceutically acceptable salt,” as use herein, includes those salts which are, within the scope of sound medical judgment, suitable for use in contact with the tissues of humans and animals without undue toxicity, irritation, allergic response and the like and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds of the invention or separately by reacting the free base group with a suitable organic or inorganic acid. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like.

The terms “pharmaceutically acceptable solvate” or “solvate,” as used herein, includes a compound of the invention wherein molecules of a suitable solvent are incorporated in the crystal lattice. A suitable solvent is physiologically tolerable at the administered dose. For example, solvates may be prepared by crystallization, recrystallization, or precipitation from a solution that includes organic solvents, water, or a mixture thereof. Examples of suitable solvents are ethanol, water (for example, mono-, di-, and tri-hydrates), N-methylpyrrolidinone (NMP), dimethyl sulfoxide (DMSO), N,N′-dimethylformamide (DMF), N,N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the solvate is referred to as a “hydrate.”

The term “prevent,” as used herein, includes prophylactic treatment or treatment that prevents one or more symptoms or conditions of a disease, disorder, or conditions described herein (e.g., CVD). Treatment can be initiated, for example, prior to (“pre-exposure prophylaxis”) or following (“post-exposure prophylaxis”) an event that precedes the onset of the disease, disorder, or conditions. Treatment that includes administration of a compound of the invention, or a pharmaceutical composition thereof, can be acute, short-term, or chronic. The doses administered may be varied during the course of preventive treatment.

The term “prodrug,” as used herein, includes compounds which are rapidly transformed in vivo to the parent compound of the above formula. Prodrugs also encompass bioequivalent compounds that, when administered to a human, lead to the in vivo formation of therapeutic. A thorough discussion is provided in T. Higuchi and V. Stella, Pro-drugs as Novel Delivery Systems, Vol. 14 of the A.C.S. Symposium Series, and Edward B. Roche, ed., Bioreversible Carriers in Drug Design, American Pharmaceutical Association and Pergamon Press, 1987, each of which is incorporated herein by reference. Preferably, prodrugs of the compounds of the present invention are pharmaceutically acceptable.

As used herein, and as well understood in the art, “treatment” includes an approach for obtaining beneficial or desired results, such as clinical results. Beneficial or desired results can include, but are not limited to, alleviation or amelioration of one or more symptoms or conditions; diminishment of extent of disease, disorder, or condition; stabilized (i.e. not worsening) state of disease, disorder, or condition; preventing spread of disease, disorder, or condition; delay or slowing the progress of the disease, disorder, or condition; amelioration or palliation of the disease, disorder, or condition; and remission (whether partial or total), whether detectable or undetectable. “Treatment” can also mean prolonging survival as compared to expected survival if not receiving treatment. As used herein, the terms “treating” and “treatment” can also include delaying the onset of, impeding or reversing the progress of, or alleviating either the disease or condition to which the term applies, or one or more symptoms of such disease or condition.

The term “unit dosage forms” includes physically discrete units suitable as unitary dosages for human subjects and other mammals, each unit containing a predetermined quantity of active material calculated to produce the desired therapeutic effect, in association with any suitable pharmaceutical excipient or excipients.

As used herein, the term “plasma concentration” includes the amount of therapeutic present in the plasma of a treated subject (e.g., as measured in a rabbit using an assay described below or in a human).

### Pharmaceutical Compositions

The methods described herein can also include the administrations of pharmaceutically acceptable compositions that include the therapeutic, or a pharmaceutically acceptable salt, solvate, or prodrug thereof. When employed as pharmaceuticals, any of the present compounds can be administered in the form of pharmaceutical compositions. These compositions can be prepared in a manner well known in the pharmaceutical art, and can be administered by a variety of routes, depending upon whether local or systemic treatment is desired and upon the area to be treated. Administration may be topical, parenteral, intravenous, intra-arterial, subcutaneous, intramuscular, intracranial, intraorbital, ophthalmic, intraventricular, intracapsular, intraspinal, intracisternal, intraperitoneal, intranasal, aerosol, by suppositories, or oral administration.

This invention also includes pharmaceutical compositions which can contain one or more pharmaceutically acceptable carriers. In making the pharmaceutical compositions of the invention, the active ingredient is typically mixed with an excipient, diluted by an excipient or enclosed within such a carrier in the form of, for example, a capsule, sachet, paper, or other container. When the excipient serves as a diluent, it can be a solid, semisolid, or liquid material (e.g., normal saline), which acts as a vehicle, carrier or medium for the active ingredient. Thus, the compositions can be in the form of tablets, powders, lozenges, sachets, cachets, elixirs, suspensions, emulsions, solutions, syrups, and soft and hard gelatin capsules. As is known in the art, the type of diluent can vary depending upon the intended route of administration. The resulting compositions can include additional agents, such as preservatives.

The therapeutic agents of the invention can be administered alone, or in a mixture, in the presence of a pharmaceutically acceptable excipient or carrier. The excipient or carrier is selected on the basis of the mode and route of administration. Suitable pharmaceutical carriers, as well as pharmaceutical necessities for use in pharmaceutical formulations, are described in Remington: The Science and Practice of Pharmacy, 22^{nd} Ed., Gennaro, Ed., Lippencott Williams & Wilkins (2012), a well-known reference text in this field, and in the USP/NF (United States Pharmacopeia and the National Formulary), each of which is incorporated by reference. In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g. about 40 mesh.

Examples of suitable excipients are lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. Other exemplary excipients are described in Handbook of Pharmaceutical Excipients, 8^{th} Edition, Sheskey et al., Eds., Pharmaceutical Press (2017), which is incorporated by reference.

The methods described herein can include the administration of a therapeutic, or prodrugs or pharmaceutical compositions thereof, or other therapeutic agents. Exemplary therapeutics include those that decrease blood pressure, control blood sugar, prevent blood clots, slow heart rate, dilate arteries, and/or increase NO concentration, for example.

The pharmaceutical compositions can be formulated so as to provide immediate, extended, or delayed release of the active ingredient after administration to the patient by employing procedures known in the art.

The compositions can be formulated in a unit dosage form, each dosage containing, e.g., 0.1-500 mg of the active ingredient. For example, the dosages can contain from about 0.1 mg to about 50 mg, from about 0.1 mg to about 40 mg, from about 0.1 mg to about 20 mg, from about 0.1 mg to about 10 mg, from about 0.2 mg to about 20 mg, from about 0.3 mg to about 15 mg, from about 0.4 mg to about 10 mg, from about 0.5 mg to about 1 mg; from about 0.5 mg to about 100 mg, from about 0.5 mg to about 50 mg, from about 0.5 mg to about 30 mg, from about 0.5 mg to about 20 mg, from about 0.5 mg to about 10 mg, from about 0.5 mg to about 5 mg; from about 1 mg from to about 50 mg, from about 1 mg to about 30 mg, from about 1 mg to about 20 mg, from about 1 mg to about 10 mg, from about 1 mg to about 5 mg; from about 5 mg to about 50 mg, from about 5 mg to about 20 mg, from about 5 mg to about 10 mg; from about 10 mg to about 100 mg, from about 20 mg to about 200 mg, from about 30 mg to about 150 mg, from about 40 mg to about 100 mg, from about 50 mg to about 100 mg of the active ingredient, from about 50 mg to about 300 mg, from about 50 mg to about 250 mg, from about 100 mg to about 300 mg, or, from about 100 mg to about 250 mg of the active ingredient. For preparing solid compositions such as tablets, the principal active ingredient is mixed with one or more pharmaceutical excipients to form a solid bulk formulation composition containing a homogeneous mixture of a compound of the present invention. When referring to these bulk formulation compositions as homogeneous, the active ingredient is typically dispersed evenly throughout the composition so that the composition can be readily subdivided into equally effective unit dosage forms such as tablets and capsules. This solid bulk formulation is then subdivided into unit dosage forms of the type described above containing from, for example, 0.1 to about 500 mg of the active ingredient of the present invention.

Compositions for Oral Administration

The pharmaceutical compositions contemplated by the invention include those formulated for oral administration (“oral dosage forms”). Oral dosage forms can be, for example, in the form of tablets, capsules, a liquid solution or suspension, a powder, or liquid or solid crystals, which contain the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

Formulations for oral administration may also be presented as chewable tablets, as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

Controlled release compositions for oral use may be constructed to release the active drug by controlling the dissolution and/or the diffusion of the active drug substance. Any of a number of strategies can be pursued in order to obtain controlled release and the targeted plasma concentration vs time profile. In one example, controlled release is obtained by appropriate selection of various formulation parameters and ingredients, including, e.g., various types of controlled release compositions and coatings. Thus, the drug is formulated with appropriate excipients into a pharmaceutical composition that, upon administration, releases the drug in a controlled manner. Examples include single or multiple unit tablet or capsule compositions, oil solutions, suspensions, emulsions, microcapsules, microspheres, nanoparticles, patches, and liposomes. In certain embodiments, compositions include biodegradable, pH, and/or temperature-sensitive polymer coatings.

Dissolution or diffusion controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palm itostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

The liquid forms in which the compounds and compositions of the present invention can be incorporated for administration orally include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions suitable for oral mucosal administration (e.g., buccal or sublingual administration) include tablets, lozenges, and pastilles, where the active ingredient is formulated with a carrier, such as sugar, acacia, tragacanth, or gelatin and glycerine.

Coatings

The pharmaceutical compositions formulated for oral delivery, such as tablets or capsules of the present invention can be coated or otherwise compounded to provide a dosage form affording the advantage of delayed or extended release. The coating may be adapted to release the active drug substance in a predetermined pattern (e.g., in order to achieve a controlled release formulation) or it may be adapted not to release the active drug substance until after passage of the stomach, e.g., by use of an enteric coating (e.g., polymers that are pH-sensitive (“pH controlled release”), polymers with a slow or pH-dependent rate of swelling, dissolution or erosion (“time-controlled release”), polymers that are degraded by enzymes (“enzyme-controlled release” or “biodegradable release”) and polymers that form firm layers that are destroyed by an increase in pressure (“pressure-controlled release”)). Exemplary enteric coatings that can be used in the pharmaceutical compositions described herein include sugar coatings, film coatings (e.g., based on hydroxypropyl methylcellulose, methylcellulose, methyl hydroxyethylcellulose, hydroxypropylcellulose, carboxymethylcellulose, acrylate copolymers, polyethylene glycols and/or polyvinylpyrrolidone), or coatings based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose. Furthermore, a time delay material such as, for example, glyceryl monostearate or glyceryl distearate, may be employed.

For example, the tablet or capsule can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release.

When an enteric coating is used, desirably, a substantial amount of the drug is released in the lower gastrointestinal tract.

In addition to coatings that effect delayed or extended release, the solid tablet compositions may include a coating adapted to protect the composition from unwanted chemical changes (e.g., chemical degradation prior to the release of the active drug substance). The coating may be applied on the solid dosage form in a similar manner as that described in Encyclopedia of Pharmaceutical Technology, vols. 5 and 6, Eds. Swarbrick and Boyland, 2000.

Parenteral Administration

Within the scope of the present invention are also parenteral depot systems from biodegradable polymers. These systems are injected or implanted into the muscle or subcutaneous tissue and release the incorporated drug over extended periods of time, ranging from several days to several months. Both the characteristics of the polymer and the structure of the device can control the release kinetics which can be either continuous or pulsatile. Polymer-based parenteral depot systems can be classified as implants or microparticles. The former are cylindrical devices injected into the subcutaneous tissue whereas the latter are defined as spherical particles in the range of 10-100 μm. Extrusion, compression or injection molding are used to manufacture implants whereas for microparticles, the phase separation method, the spray-drying technique and the water-in-oil-in-water emulsion techniques are frequently employed. The most commonly used biodegradable polymers to form microparticles are polyesters from lactic and/or glycolic acid, e.g. poly(glycolic acid) and poly(L-lactic acid) (PLG/PLA microspheres). Of particular interest are in situ forming depot systems, such as thermoplastic pastes and gelling systems formed by solidification, by cooling, or due to the sol-gel transition, cross-linking systems and organogels formed by amphiphilic lipids. Examples of thermosensitive polymers used in the aforementioned systems include, N-isopropylacrylamide, poloxamers (ethylene oxide and propylene oxide block copolymers, such as poloxamer 188 and 407), poly(N-vinyl caprolactam), poly(siloethylene glycol), polyphosphazenes derivatives and PLGA-PEG-PLGA.

Mucosal Drug Delivery

Mucosal drug delivery (e.g., drug delivery via the mucosal linings of the nasal, rectal, vaginal, ocular, or oral cavities) can also be used in the methods described herein. Methods for oral mucosal drug delivery include sublingual administration (via mucosal membranes lining the floor of the mouth), buccal administration (via mucosal membranes lining the cheeks), and local delivery (Harris et al., Journal of Pharmaceutical Sciences, 81(1): 1-10, 1992).

Oral transmucosal absorption is generally rapid because of the rich vascular supply to the mucosa and allows for a rapid rise in blood concentrations of the therapeutic.

For buccal administration, the compositions may take the form of, e.g., tablets, lozenges, etc. formulated in a conventional manner. Permeation enhancers can also be used in buccal drug delivery. Exemplary enhancers include 23-lauryl ether, aprotinin, azone, benzalkonium chloride, cetylpyridinium chloride, cetyltrimethylammonium bromide, cyclodextrin, dextran sulfate, lauric acid, lysophosphatidylcholine, methol, methoxysalicylate, methyloleate, oleic acid, phosphatidylcholine, polyoxyethylene, polysorbate 80, sodium EDTA, sodium glycholate, sodium glycodeoxycholate, sodium lauryl sulfate, sodium salicylate, sodium taurocholate, sodium taurodeoxycholate, sulfoxides, and alkyl glycosides. Bioadhesive polymers have extensively been employed in buccal drug delivery systems and include cyanoacrylate, polyacrylic acid, hydroxypropyl methylcellulose, and poly methacrylate polymers, as well as hyaluronic acid and chitosan.

Liquid drug formulations (e.g., suitable for use with nebulizers and liquid spray devices and electrohydrodynamic (EHD) aerosol devices) can also be used. Other methods of formulating liquid drug solutions or suspension suitable for use in aerosol devices are known to those of skill in the art (see, e.g., Biesalski, U.S. Pat. No. 5,112,598, and Biesalski, U.S. Pat. No. 5,556,611).

Formulations for sublingual administration can also be used, including powders and aerosol formulations. Exemplary formulations include rapidly disintegrating tablets and liquid-filled soft gelatin capsules.

Dosing Regimes

The present methods for treating CVD are carried out by administering a therapeutic for a time and in an amount sufficient to result in increased blood flow or decreased pain, for example.

The amount and frequency of administration of the compositions can vary depending on, for example, what is being administered, the state of the patient, and the manner of administration. In therapeutic applications, compositions can be administered to a patient suffering from CVD in an amount sufficient to relieve or least partially relieve the symptoms of the CVD and its complications. The dosage is likely to depend on such variables as the type and extent of progression of the CVD, the severity of the CVD, the age, weight and general condition of the particular patient, the relative biological efficacy of the composition selected, formulation of the excipient, the route of administration, and the judgment of the attending clinician. Effective doses can be extrapolated from dose-response curves derived from in vitro or animal model test system. An effective dose is a dose that produces a desirable clinical outcome by, for example, improving a sign or symptom of CVD or slowing its progression.

The amount of therapeutic per dose can vary. For example, a subject can receive from about 0.1 μg/kg to about 10,000 μg/kg. Generally, the therapeutic is administered in an amount such that the peak plasma concentration ranges from 150 nM-250 μM.

Exemplary dosage amounts can fall between 0.1-5000 μg/kg, 100-1500 μg/kg, 100-350 μg/kg, 340-750 μg/kg, or 750-1000 μg/kg. Exemplary dosages can 0.25, 0.5, 0.75, 1°, or 2 mg/kg. In another embodiment, the administered dosage can range from 0.05-5 mmol of therapeutic (e.g., 0.089-3.9 mmol) or 0.1-50 μmol of therapeutic (e.g., 0.1-25 μmol or 0.4-20 μmol).

The plasma concentration of therapeutic can also be measured according to methods known in the art. Exemplary peak plasma concentrations of therapeutic can range from 0.05-10 μM, 0.1-10 μM, 0.1-5.0 μM, or 0.1-1 μM. Alternatively, the average plasma levels of therapeutic can range from 400-1200 μM (e.g., between 500-1000 μM) or between 50-250 μM (e.g., between 40-200 μM). In some embodiments where sustained release of the drug is desirable, the peak plasma concentrations (e.g., of therapeutic) may be maintained for 6-14 hours, e.g., for 6-12 or 6-10 hours. In other embodiments where immediate release of the drug is desirable, the peak plasma concentration (e.g., of therapeutic) may be maintained for, e.g., 30 minutes.

The frequency of treatment may also vary. The subject can be treated one or more times per day with therapeutic (e.g., once, twice, three, four or more times) or every so-many hours (e.g., about every 2, 4, 6, 8, 12, or 24 hours). Preferably, the pharmaceutical composition is administered 1 or 2 times per 24 hours. The time course of treatment may be of varying duration, e.g., for two, three, four, five, six, seven, eight, nine, ten or more days. For example, the treatment can be twice a day for three days, twice a day for seven days, twice a day for ten days. Treatment cycles can be repeated at intervals, for example weekly, bimonthly or monthly, which are separated by periods in which no treatment is given. The treatment can be a single treatment or can last as long as the life span of the subject (e.g., many years).

Kits

Any of the pharmaceutical compositions of the invention described herein can be used together with a set of instructions, i.e., to form a kit. The kit may include instructions for use of the pharmaceutical compositions as a therapy as described herein. For example, the instructions may provide dosing and therapeutic regimes for use of the compounds of the invention to reduce symptoms and/or underlying cause of the CVD. Additionally, diagnosis kits may be provided which measure one, two, or all of the plasma sulfide pools and aid in diagnosing CVD, CAD, and/or PAD.

Various objects, features, aspects, and advantages of the present invention will become more apparent from the following detailed description of preferred embodiments of the invention, along with the accompanying drawings in which like numerals represent like components. The present invention may address one or more of the problems and deficiencies of the current technology discussed above. However, it is contemplated that the invention may prove useful in addressing other problems and deficiencies in a number of technical areas. Therefore the claimed invention should not necessarily be construed as limited to addressing any of the particular problems or deficiencies discussed herein.

### BRIEF DESCRIPTION OF THE DRAWINGS

The accompanying drawings, which are incorporated in and constitute a part of the specification, illustrate various embodiments of the invention and together with the general description of the invention given above and the detailed description of the drawings given below, serve to explain the principles of the invention. It is to be appreciated that the accompanying drawings are not necessarily to scale since the emphasis is instead placed on illustrating the principles of the invention. The invention will now be described, by way of example, with reference to the accompanying drawings in which:

FIG. 1 is a Study organization flow chart. A total of 324 patients were used for analysis, including 209 males and 115 females. This subject population was subsequently diagnosed with or without CAD or PAD after cardiac catheterization. CAD indicates coronary artery disease; PAD, peripheral artery disease.

FIG. 2 is a set of nine graphs showing plasma sulfide bioavailability by ethnicity. Total sulfide, acid labile pools, and bound sulfide in total subject populations (combined), Caucasian and African American subjects respectively with any form of CVD. CVD indicates cardiovascular disease. Control Caucasian vs African Americans ### p=0.0001; #p=0.0028; ##p=0.0326.

FIG. 3 is a set of nine graphs showing plasma sulfide pools in women by ethnicity. Total sulfide, acid labile pools, and bound sulfide levels of Plasma have been displayed in female subjects with and without any CVD. CVD indicates cardiovascular disease. Control Caucasian vs African Americans ###p=0.0031; #p=0.06555.

FIG. 4 is a set of nine graphs showing plasma sulfide pools in men by ethnicity. Total sulfide, acid labile pools, and bound sulfide levels of Plasma have been displayed in male subjects with and without any CVD. CVD indicates cardiovascular disease. Control Caucasian vs African Americans ###p=0.0338; #p=0.03085.p=0.0338; #p=0.03085.

FIG. 5 is a set of nine graphs showing sulfide bioavailability in CAD and PAD subjects. Plasma total, acid labile and bound sulfane sulfide pools in a combined or total subject population, Caucasian and African American subjects with either CAD or PAD. CAD indicates Coronary arterial disease; PAD, Peripheral arterial disease. Control Caucasian vs African Americans ###p=0.0001; #p=0.0028; ##p=0.0326.

FIGS. 6A-6F are a set of six line graphs showing receiver-operating characteristic analysis (ROC) in subjects with CVD. The ROC curves with area under the curve of Caucasian population. FIG. 6A shows total sulfide levels in females. FIG. 6B shows total sulfide levels in males. FIG. 6C shows Acid labile pools in females. FIG. 6D shows Bound sulfane sulfur in males. FIG. 6E shows total sulfide. FIG. 6F shows total sulfide levels in African American CVD patients. CVD indicates cardiovascular disease.

FIG. 7 is a table listing Subject Demographics. AA=African American; Cauc=Caucasian; PAD=Peripheral Arterial Disease; CAD=Coronary Artery Disease; DM=Diabetes Mellitus; HTN=Hypertension; BMI=Body Mass Index.

FIG. 8 is a table showing a regression analysis of CVD risk factors for all subjects.

FIG. 9 is a table showing a regression analysis of CVD risk factors for Caucasian subjects.

FIG. 10 is a table showing a regression analysis of CVD risk factors for African American subjects.

FIG. 11 is a table listing mutant allele frequencies of CTH, CBS, and NOS3 single-nucleotide polymorphisms. CTH: Cystathionine γ-lyase; CBS: cystathionine β-synthase; NOS3: endothelial NO synthase; PAD: Peripheral artery disease; CAD: Coronary Artery Disease; CVD: Cardiovascular Disease (PAD and/or CAD); N/A: not applicable—mutant allele not detected within cohort.

FIG. 12 is a table listing Plasma Free Sulfide Levels of various subject demographics.

### DETAILED DESCRIPTION

The present invention will be understood by reference to the following detailed description, which should be read in conjunction with the appended drawings. It is to be appreciated that the following detailed description of various embodiments is by way of example only and is not meant to limit, in any way, the scope of the present invention. In the summary above, in the following detailed description, in the claims below, and in the accompanying drawings, reference is made to particular features (including method steps) of the present invention. It is to be understood that the disclosure of the invention in this specification includes all possible combinations of such particular features, not just those explicitly described. For example, where a particular feature is disclosed in the context of a particular aspect or embodiment of the invention or a particular claim, that feature can also be used, to the extent possible, in combination with and/or in the context of other particular aspects and embodiments of the invention, and in the invention generally. The term “comprises” and grammatical equivalents thereof are used herein to mean that other components, ingredients, steps, etc. are optionally present. For example, an article “comprising” (or “which comprises”) components A, B, and C can consist of (i.e., contain only) components A, B, and C, or can contain not only components A, B, and C but also one or more other components. Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

The term “at least” followed by a number is used herein to denote the start of a range beginning with that number (which may be a range having an upper limit or no upper limit, depending on the variable being defined). For example “at least 1” means 1 or more than 1. The term “at most” followed by a number is used herein to denote the end of a range ending with that number (which may be a range having 1 or 0 as its lower limit, or a range having no lower limit, depending upon the variable being defined). For example, “at most 4” means 4 or less than 4, and “at most 40%” means 40% or less than 40%. When, in this specification, a range is given as “(a first number) to (a second number)” or “(a first number)-(a second number),” this means a range whose lower limit is the first number and whose upper limit is the second number. For example, 25 to 100 mm means a range whose lower limit is 25 mm, and whose upper limit is 100 mm. The embodiments set forth the below represent the necessary information to enable those skilled in the art to practice the invention and illustrate the best mode of practicing the invention. In addition, the invention does not require that all the advantageous features and all the advantages need to be incorporated into every embodiment of the invention.

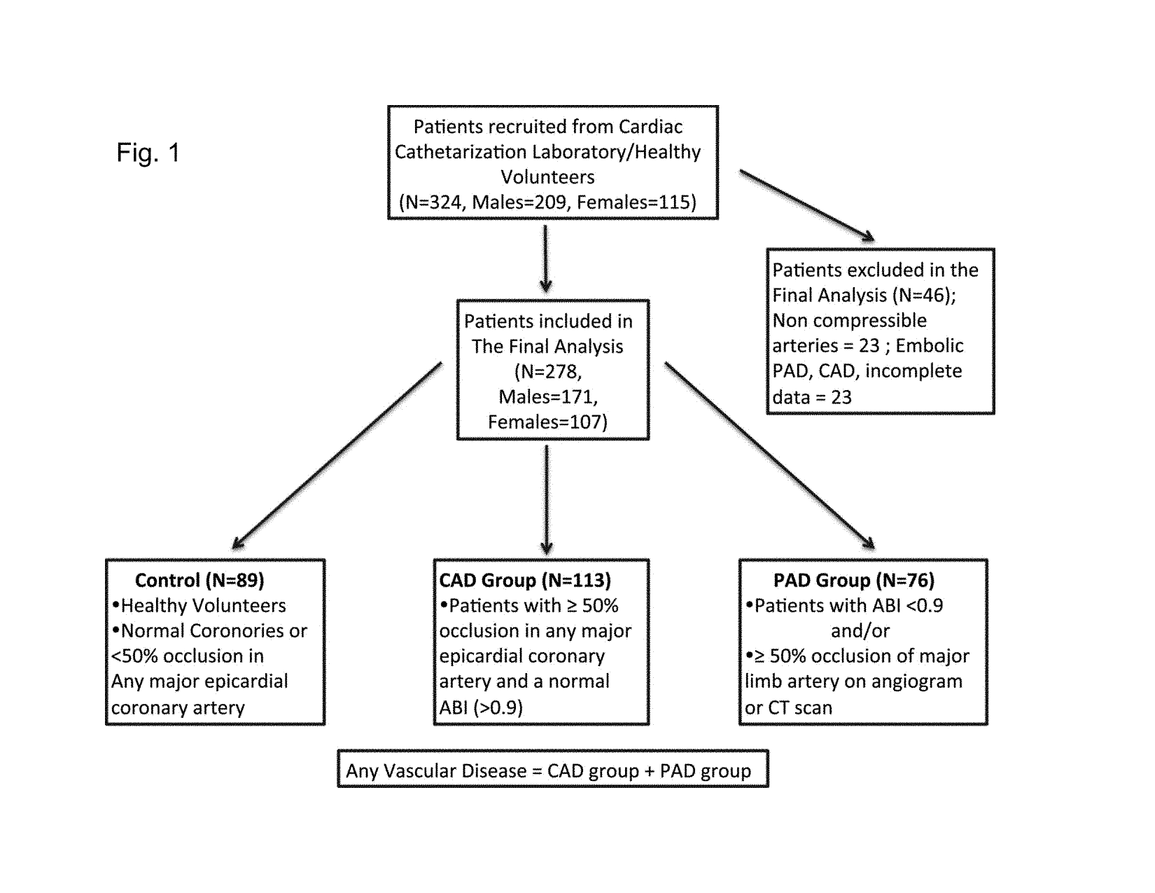

Turning now to FIGS. 1-12, a brief description concerning the various components of the present invention will now be briefly discussed.

With recognition and definition of physiologic effects of nitric oxide, the inventors have been interested in the biological activity of the “other” gaseous signaling molecules, namely hydrogen sulfide (H2S) and carbon monoxide (CO). H2S is produced endogenously via enzymes of the transsulfuration pathway including cystathionine β-synthase (CBS) and cystathionine γ-lyase (CGL, CTH or CSE), as well as the mitochondrial enzyme 3-mercaptopyruvate sulfurtransferase (MST). H2S may also be generated through a non-enzymatic process from glucose (via glycolysis, NADPH oxidase), glutathione (direct reduction), inorganic and organic polysulfides (present in foods) or through elemental sulfur (direct reduction). Alteration of H2S bioavailability and metabolism through many of these pathways influences cardiovascular function and health. Unfortunately, the relationship of H2S bioavailability with clinical cardiovascular disease conditions remains poorly defined in the art.

Atherosclerotic cardiovascular disease is still the most common and costly cause of death in the United States and much of the world. Chronic vascular inflammation and sub-endothelial accumulation of foam cells stimulate occlusion and stenosis of blood vessels, which is a common culprit underlying peripheral and coronary arterial disease. Metabolic dysfunction involving reduced production of cellular H2S may be a critical factor in the progression of experimental cardiovascular disease. Endogenous H2S production is significantly reduced in the CTH (CSE) knockout mouse model, which is associated with impaired endothelial vasodilation and hypertension, increased production of reactive oxygen species, increased vascular inflammatory responses, and enhanced vascular atherosclerosis. Further, H2S therapy using sulfide donors diminishes vascular inflammatory responses, decrease reactive oxygen species, and promote ischemic vascular remodeling/angiogenesis involving increased NO production.

H2S exists in different biochemical forms, including free or unbound sulfide (S2-, HS- or H2S), acid labile sulfide (ALS), and bound sulfane sulfur (BSS). These sulfur pools are crucial in regulating the total amount of bioavailable sulfide. ALS exists primarily in the form of iron-sulfur (Fe—S) complexes that modulate cellular functions including mitochondrial respiration and cytoplasmic redox reactions. BSS includes various compounds such as persulfides, polysulfides, thiosulfate, polythionates, thiosulfonates, bisorganylpolysulfanes or monoarylthiosulfonates, elemental sulfur, and many others. BSS compounds such as per/polysulfides can release H2S under reducing conditions suggesting that the cellular redox state is important for regulating its bioavailability. The precise chemistry through which these different biological pools of H2S interact to affect their pathophysiological functions is an area of active research. However, differences in bioavailability of these biochemical pools of sulfide remain largely unknown in the art in part due to difficulties in measuring them. Overall, the sulfide field has been limited by controversies related to measurements of H2S in various biological systems. The inventors' lab has established and validated analytical chemistry methods to accurately detect and quantify discrete H2S pools using a monobromobimane (MBB) assay coupled with reverse-phase high performance liquid chromatography (RP-HPLC), which was verified by electrospray ionization mass spectrometry.

### Materials & Methods

Study design: This was a case-control study approved by the Institutional Review Board (IRB) of Louisiana State University Health Sciences Center at Shreveport (LSUHSC-S). Patients over 40 years of age who presented to the cardiac catheterization laboratory at LSUHSC-S for coronary or peripheral angiography were recruited for this study. Healthy, age-matched volunteers were also enrolled as controls. Each patient's ankle brachial index (ABI) was measured and each patient was also administered the San Diego Claudication Questionnaire prior to angiography. Following exclusion criteria, the total study population consisted of 278 Caucasian and African American (AA) subjects categorized into the following three basic subgroups (See FIG. 1).

Healthy Controls:

healthy volunteers and patients with less than 50% occlusion of all major coronary or peripheral arteries and a normal ABI (1.4>ABI>0.9).

Coronary Arterial Disease (CAD):

patients with greater than or equal to 50% occlusion of any major coronary artery and a normal ABI (1.4>ABI>0.9).

Peripheral Arterial Disease (PAD):

patients with greater than 50% occlusion of a major limb artery and/or an abnormal ABI (ABI<0.9).

Exclusion Criteria:

Volunteers who were excluded from this study were those who could not provide informed consent, were participating in another clinical trial involving experimental therapeutics, or were pregnant or nursing. Patients with ST elevated myocardial infarction or cardiogenic shock were not included to avoid interference with time-sensitive revascularization and confusion of pathophysiological events. Additional exclusion criteria included patients with an ABI>1.4 (due to non-compressible arteries) or patients with Buerger's disease (non-atherosclerotic PAD).

History and Blood Collection:

Patients were interviewed and medical record data were collected for analysis of typical cardiovascular risk factors such as hypertension, diabetes, obesity, tobacco use, and dyslipidemia. Blood samples were collected from already-established catheterization into 6 mL BD vacutainer tubes with lithium heparin. Samples were transported to the lab within 15 min on ice and were centrifuged at 1500 RCF for 4 min at 4° C.

Measurement of Biological Pools of H2S:

Plasma samples were analyzed for free sulfide, ALS, BSS, and total sulfide levels. Free sulfide was measured using the MBB method. For detection of ALS and BSS, 50 μl of plasma was added separately into two sets of 4 mL BD vacutainer tubes. Four hundred fifty microliters of 100 mM phosphate buffer (pH 2.6, 0.1 mM DTPA) was added to one tube [acid labile reaction] and 450 μl of 100 mM phosphate buffer (pH 2.6, 0.1 mM DTPA) plus 1 mM TCEPwas added to the second tube [total sulfide reaction]. Following a 30-min incubation on a nutator, the reaction liquid was removed and sulfide gas subsequently trapped by adding 500 μl of 100 mM Tris-HCl buffer (pH 9.5, 0.1 mM DTPA) into the BD vacutainer tube and incubated again for 30 min on a nutator mixer. The trapping solutions were removed and sulfide levels measured using the MBB method. Determination of ALS was made by reacting plasma samples with acidic phosphate buffer alone and subsequent trapping of evolved sulfide. Measurement of BSS was determined by subtracting the acid labile value from the total sulfide protocol containing TCEP reductant treatment under acidic conditions. Total sulfide levels were directly obtained from the total sulfide reaction.

MBB Assay and RP-HPLC Detection:

Thirty microliters of reaction buffer with trapped sulfide was transferred to a PCR tube and mixed with 70 μl of H2S stabilization buffer (100 mM Tris-HCl, 0.1 mM DTPA, pH 9.5) and 50 μl MBB solution (10 mM). Samples were then incubated in a hypoxic chamber (1% 02) for 30 min at room temperature. The reaction was stopped by adding 50 μl of 200 mM sulfosalicylic acid, followed by centrifugation at 12,000 rpm for 10 min at 4° C. One hundred microliters of supernatant was collected for RP-HPLC. Ten microliters of the supernatant was transferred into the RP-HPLC system with an Agilent Eclipse XDB-C18 column (5 μm, 80 Å, 4.6 mm×250 mm) equilibrated with 15% CH3CN in water containing 0.1% (v/v) TFA for fluorescence detection (excitation: 390 nm; emission: 475 nm).

MBB and sulfide-dibimane were separated using the gradient of two mobile phases: (A) water containing 0.1% (v/v) TFA and (B) 99.9% CH3CN, 0.1% (v/v) TFA at a flow rate of 0.6 mL/min. Retention time for sulfide-dibimane is 16.5 min and MBB is 17.6 min. The amount of H2S was measured from linear plots of the HPLC peak areas of sulfide-dibimane versus standard concentration of sulfide solution.

Statistical Analysis:

Levels of ALS, BSS and total H2S in the three subgroups were first assessed by group means and standard deviations with subsequent pairwise comparison using analysis of variance (ANOVA). Multiple linear regression analysis was conducted to delineate the relationship between sulfide pools and the dependent variables including race, gender, diagnosis of CAD and PAD, and cardiovascular risk factors. Receiver-operating characteristic analysis (ROC) was conducted to assess the predictive accuracy in correlating sulfide levels with CAD or PAD diagnosis. Cutoff values for positive classification were included in the curve, with a nonparametric distribution assumption and a confidence level of 95%. All statistical analyses were performed using GraphPad Prism 5.0.

### Results

Sulfide Bioavailability Associated with Cardiovascular Disease:

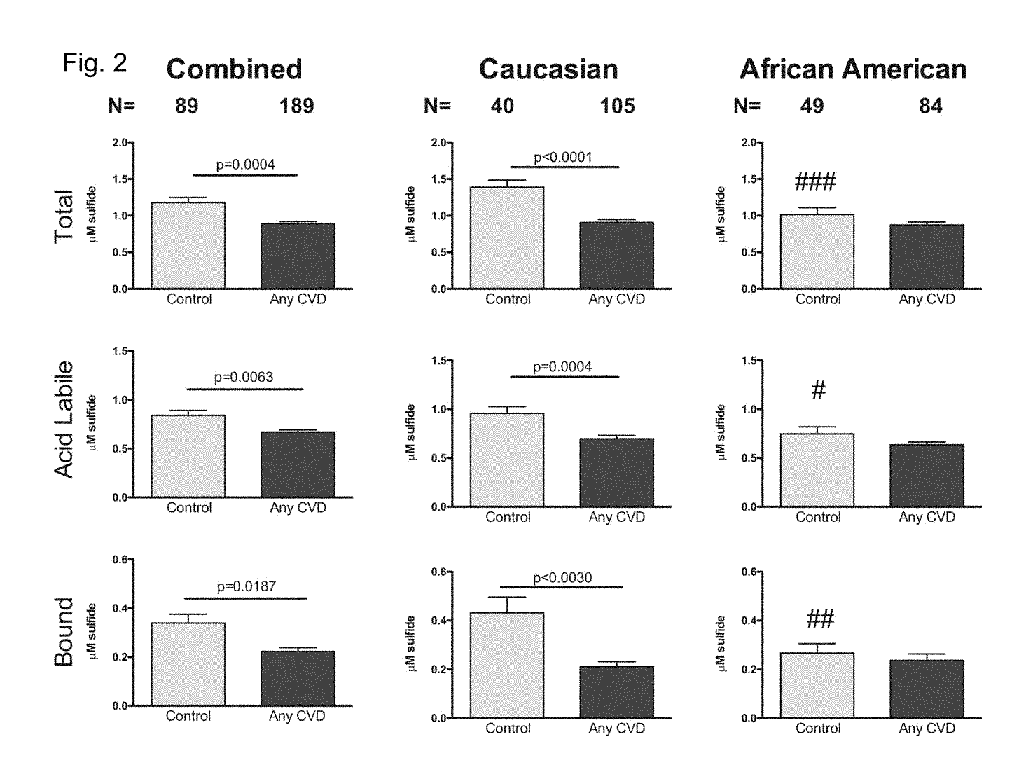

Plasma sulfide bioavailability was initially compared between control subjects and patients with any form of cardiovascular disease (any CVD). FIG. 2 illustrates that total, acid labile, and bound sulfide were all significantly reduced in patients with any CVD. Stratifying subjects based on ethnicity revealed a significant reduction in plasma total, ALS, and BSS in CVD patients compared to controls among Caucasian subjects, which was not observed in African Americans. However, comparison of control cohorts of African Americans to Caucasians revealed a significant reduction in plasma total sulfide (1.015 vs 1.389) and BSS levels (0.431 vs 0.266) in the African Americans.

Sulfide Bioavailability as a Function of Gender.

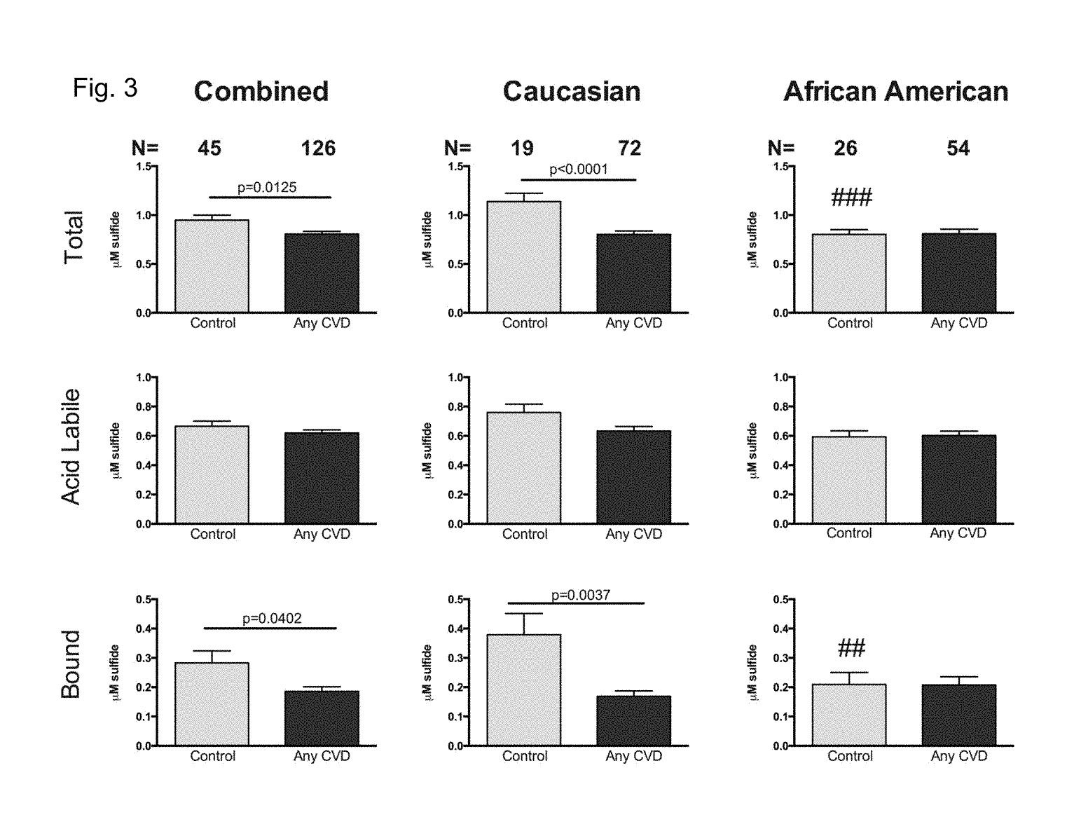

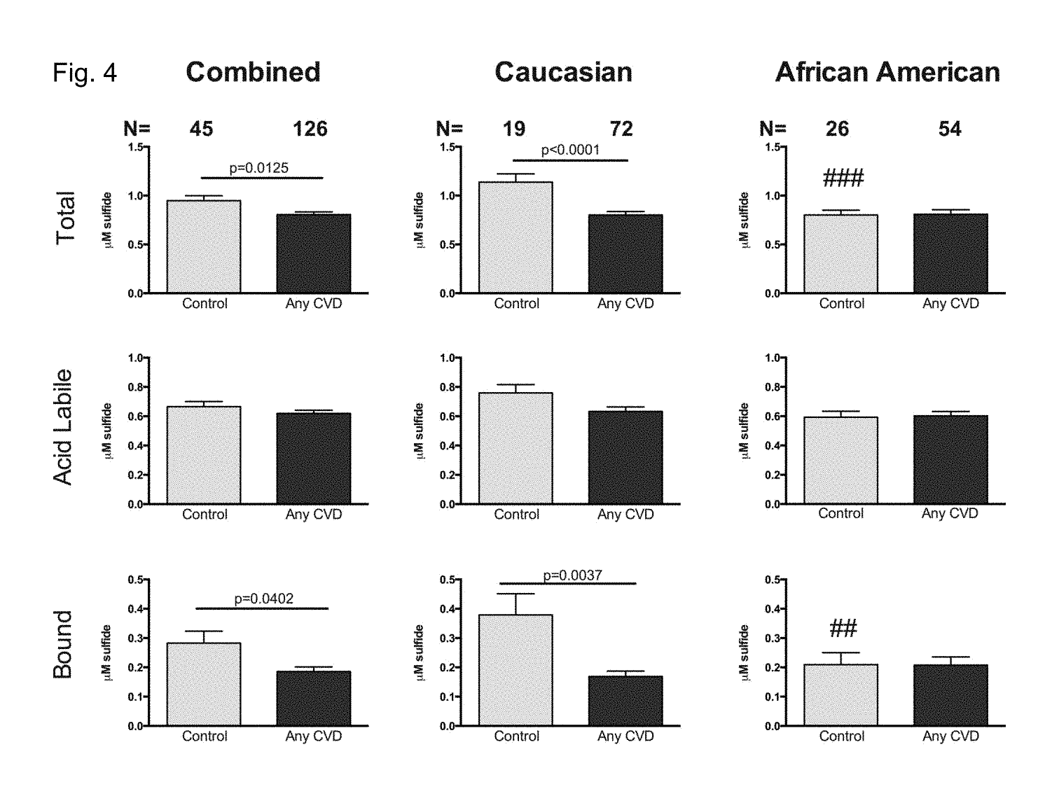

The association of plasma sulfide levels was compared between the subject population with and without CVD based on gender. FIG. 3 illustrates that women with any CVD display a significant reduction in total sulfide and ALS levels. There were no significant differences in BSS levels. Stratifying by race and gender further revealed that Caucasian female CVD patients had significantly reduced total sulfide and ALS levels compared to African American female CVD patients. FIG. 4 demonstrates that all males with any CVD had significantly reduced plasma total and BSS levels. Segregation by race and gender again revealed that plasma total and BSS levels were significantly reduced only in Caucasian males with CVD. Together, these data reveal discrete novel differences in plasma sulfide metabolites during cardiovascular disease between males and females.

Sulfide Bioavailability as a Function of Coronary or Peripheral Artery Disease:

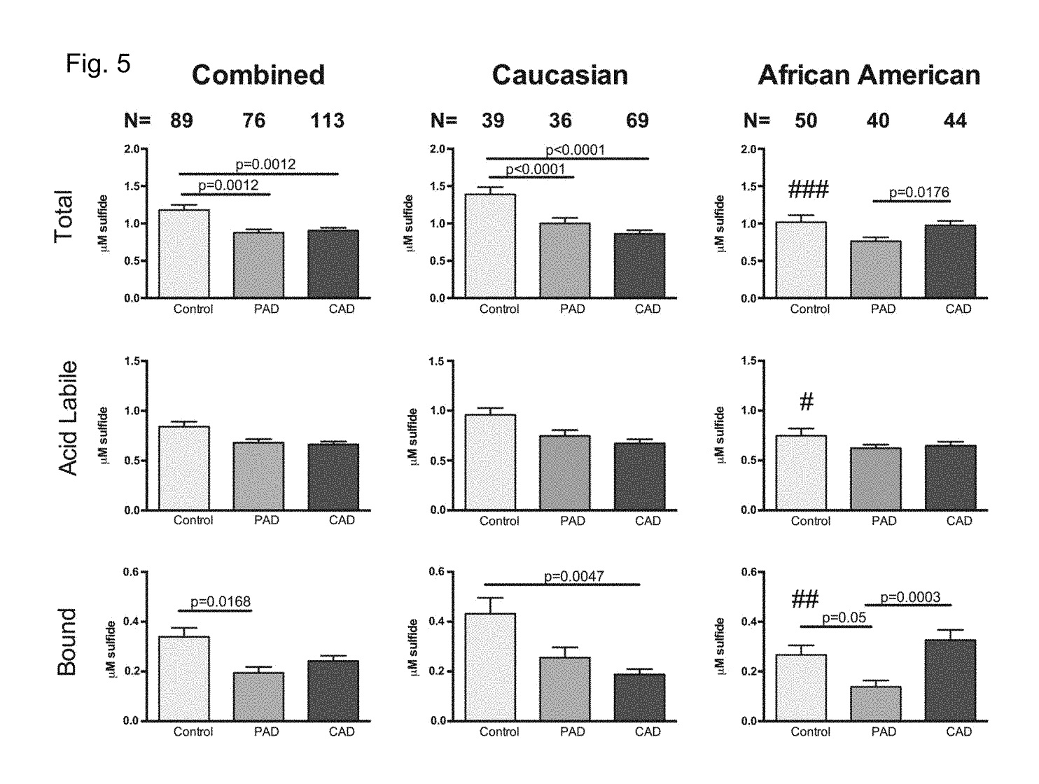

Plasma sulfide metabolite levels were next analyzed based on the diagnosis of either CAD or PAD. Importantly, a majority of patients (>85%) diagnosed with PAD were also diagnosed with CAD indicating broad CVD. Stratifying CVD subjects diagnosed as PAD or CAD revealed a significant reduction in plasma total and BSS levels, as shown in FIG. 5. Plasma total and BSS levels in Caucasian subjects with either CAD or PAD were significantly lower compared to controls, while levels of ALS were not significantly reduced. African American subjects with PAD had reduced BSS compared to controls. Conversely, BSS levels were significantly elevated in African Americans with CAD compared to PAD subjects but not that of controls. Together, these data reveal that plasma sulfide bioavailability is predominantly reduced in Caucasian subjects with either CAD or PAD. A comparison of different ethnicities between control cohorts revealed significantly reduced base levels of plasma sulfide in African Americans to Caucasians.

Sulfide as an Indicator of Cardiovascular Disease:

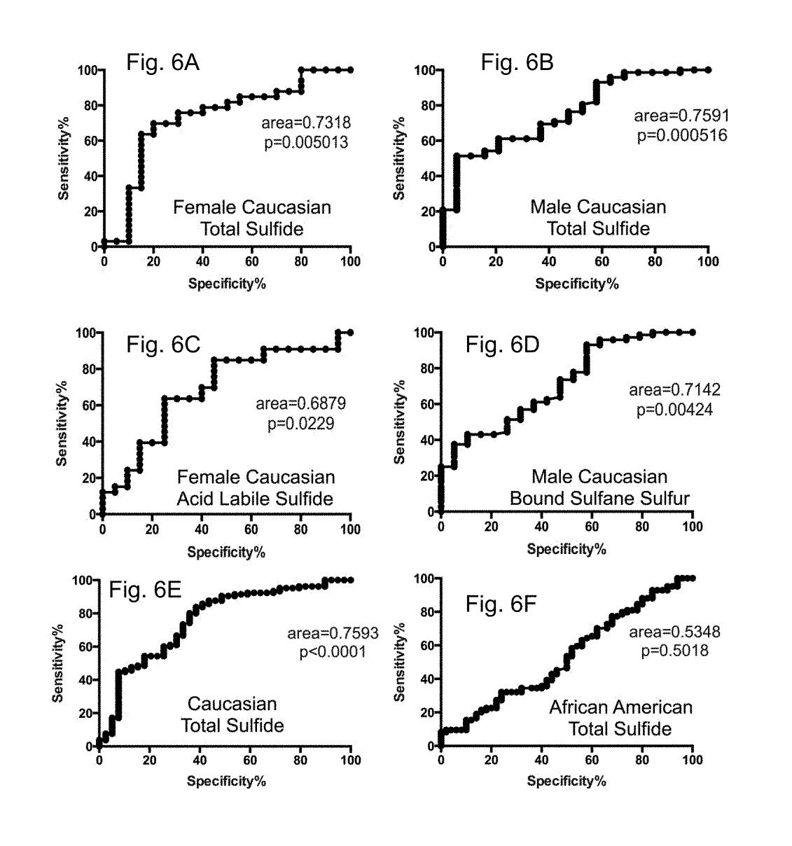

Based on the observed differences in plasma sulfide measurements between CVD patients and controls in a gender and ethnic manner, we next performed receiver operator analysis to determine the accuracy of reduced sulfide levels as an indicator for CVD. Caucasian male and female plasma total sulfide levels were analyzed separately revealing an area under the curve (AUC) of 0.7591 (p=0.0005) for males and 0.7318 (p=0.005) for females (FIGS. 6A and 6B). Having observed significant gender differences of Caucasian plasma sulfide metabolite pools, these data were further analyzed using plasma ALS in females and plasma BSS in males. FIG. 6C shows the female Caucasian ALS AUC was 0.6879 (p=0.022), whereas the male Caucasian BSS was 0.7142 (p=0.0042) (FIG. 6D). Lastly, plasma total sulfide levels were analyzed based on ethnicity and found to be a statistically significant indicator of CVD in Caucasian patients regardless of gender, with an AUC of 0.76 (p<0.0001) (FIG. 6E). However, total sulfide levels were not identified as an indicator for CVD in African American patients (FIG. 6F).

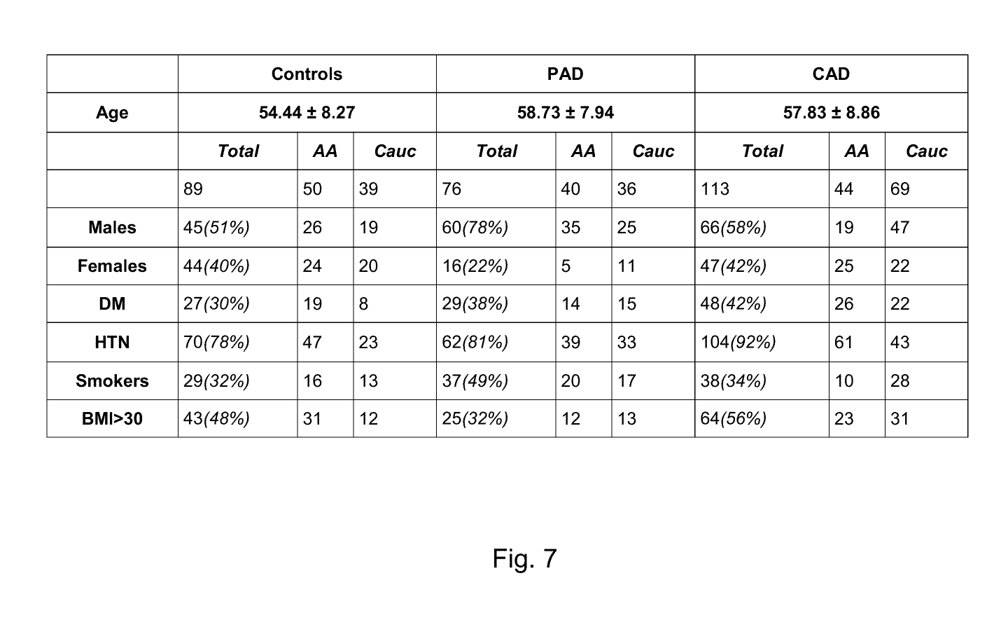

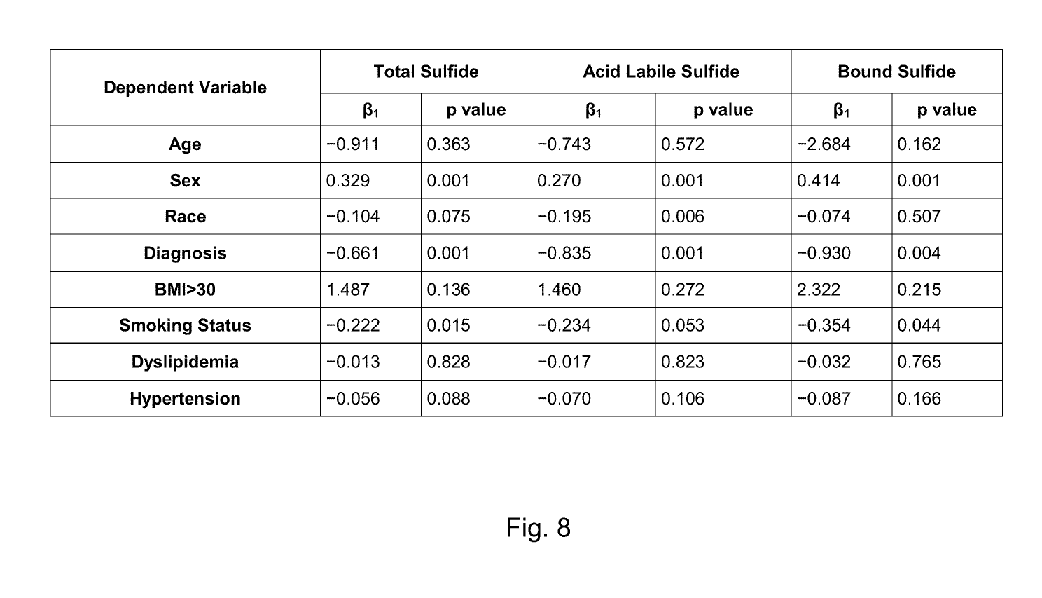

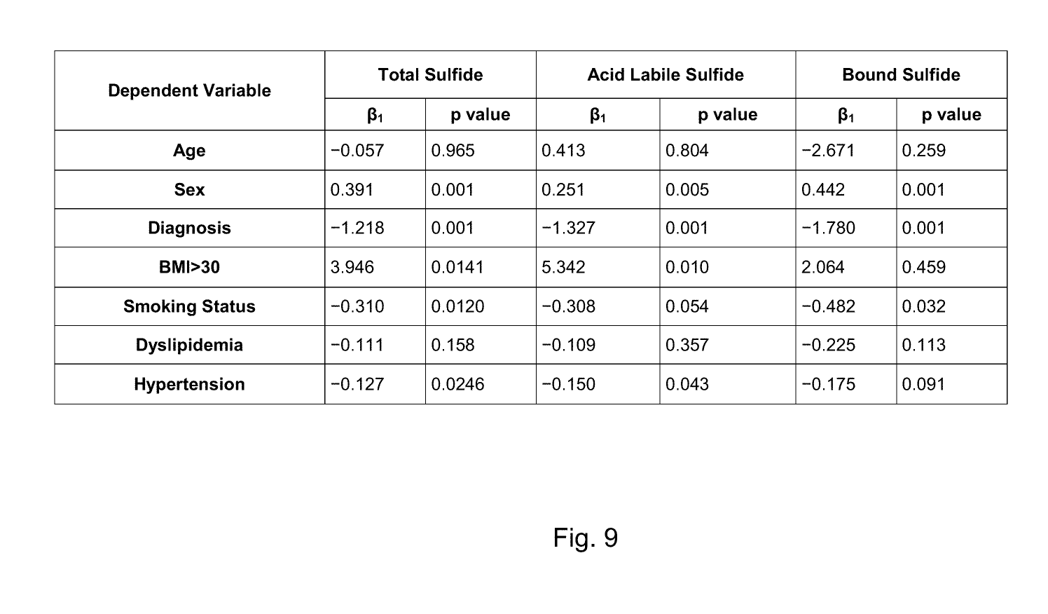

Linear regression analysis was further utilized to determine relationships between sulfide biochemical metabolite pools with various dependent variables related to cardiovascular disease including age, sex, diagnosis, BMI, tobacco use, and dyslipidemia based on subject demographics (FIG. 6). Regression analysis of CVD risk factors for all subjects revealed significant associations with ethnicity, diagnosis, and gender. Importantly, in both Caucasian and African American subjects, females were associated with higher levels of total sulfide, ALS, and BSS levels than males (FIGS. 7 and 8). The most consistent linear regression trends were seen in Caucasian subjects with cardiovascular disease (FIG. 7). Among this subgroup, total sulfide, ALS, and BSS levels were significant and most strongly associated with a diagnosis of CVD, along with other significant yet weaker associations of gender, hypertension, and smoking status. While a weak association with diagnosis was also observed in African Americans, these results were not as significant compared to Caucasians (FIGS. 8 and 9)

Single Nucleotide Polymorphisms (SNPs):

A study by Wang et al. suggests that genetic variation in cystathionine gamma-lyase (CTH) is associated with elevated plasma homocysteine levels. To further identify any such associations with CVD, the inventors screened a subset of our patient population for SNPs in three enzymes related to H2S and NO metabolism to determine if variations in allele frequencies existed between CVD patients and controls. After screening for 7 SNPs in the CTH, CBS, and eNOS (endothelial nitric oxide synthase) genes, the inventors identified two polymorphisms with considerable variation in allele frequencies among the inventors' patient population: 1 SNP in the NOS3 gene and 1 in the CTH gene.

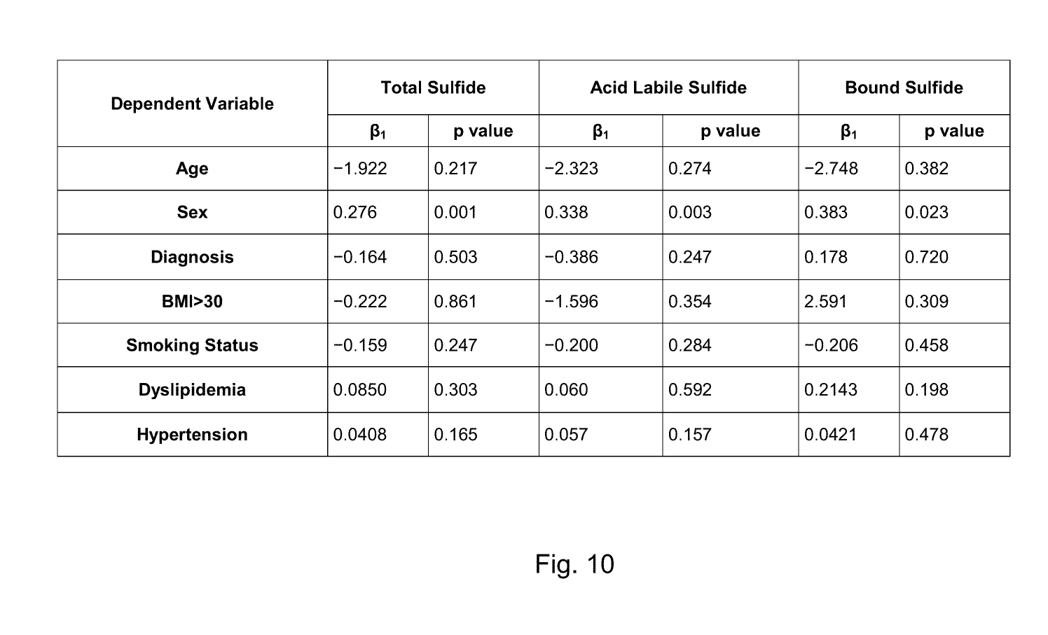

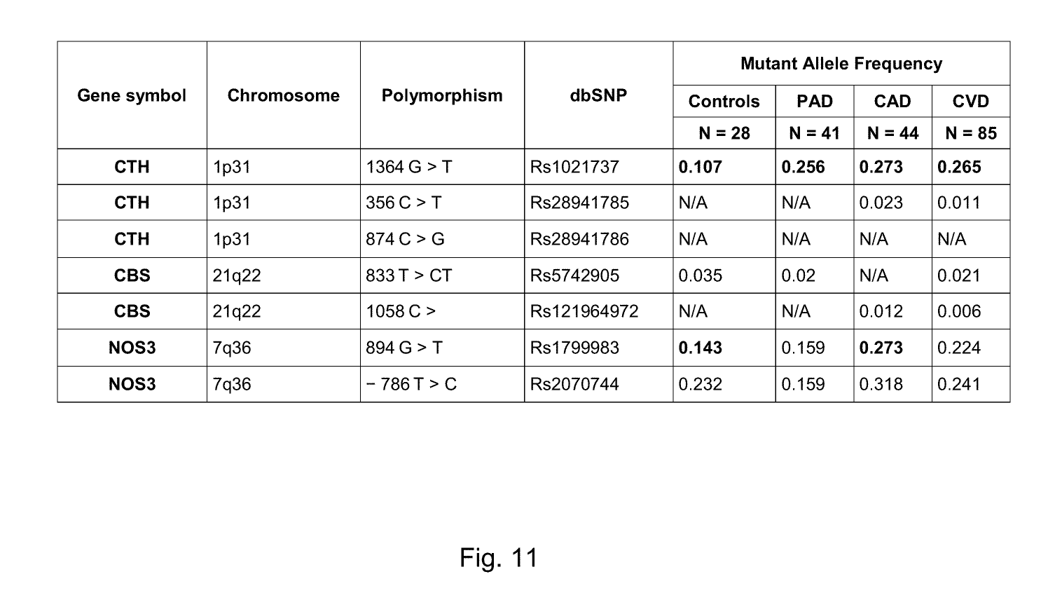

The CTH 1364 G>T SNP (Rs1021737) involves a point mutation in exon 12 of the CTH gene that was previously identified in patients with hyperhomocysteinemia. In the inventors' patient population, the mutant 1364 T allele frequency was higher in patients with CVD (0.265) and either CAD or PAD (0.256 or 0.273) compared to controls (0.107) ((FIG. 10). The inventors also identified an SNP in exon 8 of the eNOS gene (894 G>T) with considerable allelic variation among our patient population. The 894 G>T mutation (Rs1799983) has been previously identified among patients with hypertension, cerebrovascular disease, and CAD. The allele frequency of this mutation was highest in patients with CAD compared to controls (0.273 vs. 0.143, respectively; FIG. 11). These data suggest that the CTH 1364 G>T SNP is a potential risk factor with increasing mutation allele frequency with CVD including PAD and CAD, whereas eNOS 894 G>T was associated with increased risk of CAD.

### Discussion

Hydrogen sulfide metabolism and its bioavailability is associated with vascular dysfunction and disease. However, the relationship between sulfide bioavailability and clinically validated cardiovascular disease has previously remained poorly understood due to lack of a reliable and sensitive analytical measurement of sulfide in its various biochemical forms coupled with well defined, clinically validated subjects. For the first time, the inventors' study indicates an association of polysulfide to any form of vascular disease. The inventors' have herein reported the levels of total, acid labile and bound sulfane sulfide in plasma samples from clinical subjects of vascular disease using validated analytical chemistry methodology that others and we have extensively characterized. Importantly, the inventors found that subjects with CVD have lower bound and total sulfide metabolites compared to control subjects, which were observed in an ethnic and gender specific manner. The relevance of different sulfide biochemical forms including ALS and BSS represent biochemical sulfide reservoirs. These sulfide equivalents may be important under various pathophysiological conditions, which might be inter-convertible depending on pH and redox balance. Thus, the ALS and BSS pools are postulated to be a ‘reversible sulfide sink’ as free H2S is ephemeral, making it difficult to account for its prolonged physiological actions.

The biological effects of H2S are increasingly attributed to per/polysulfides that are produced endogenously in many cells and tissues of mammalian origin. These per/polysulfides can reversibly generate H2S by their degradation, which could play critical physiological roles. Additionally, conversion of a thiol to the corresponding hydropersulfide can result in a change of catalytic activity within the protein. The physiological significance of BSS is not completely understood but has recently been appreciated beyond being a storage form of sulfide. Persulfides such as alkyl hydropersulfides may be generated via H2S-independent mechanisms and can have a greater biological activity. BSS is suggested to include compounds such as per/polysulfides. However, the role of BSS in clinical complications such as cardiovascular disease has not been previously identified. Here, the inventors observed lower levels of BSS in subjects with vascular disease and those with major cardiovascular risk factors. This could be either a manifestation of the disease state itself or a “compensatory mechanism” that enables movement of sulfide to more bioavailable pools (H2S/S2-/HS-) that exert beneficial effects including anti-oxidation, vasodilation or angiogenesis. In support of the latter theory, BSS has been shown to release H2S in reducing conditions. This BSS-derived sulfide could then have biological implications that are known to be associated with H2S.

BSS also appears to be a major product of the cysteine aminotransferase and MST pathway and has been proposed to be the major pathway for sulfide signaling in the brain. H2S can be liberated from MST-BSS by the ubiquitous reductant, thioredoxin and by dihydrolipoic acid, both present in cells. Additionally, CTH and CBS can also generate BSS in tissues using thiol substrates including homocysteine, cysteine, cystathionine, and cystine, with cysteine hydropersulfide (Cys-SSH) as an intermediate. Cysteine tRNA synthase (CARS), which is involved in cysteine metabolism and aminoacyl-tRNA synthesis, is another major source of cysteinepersulfide in vivo. Interestingly, the kinetics of these enzymes vary with conditions such as oxidative stress, that influence changes in the generation of reactive persulfide species.

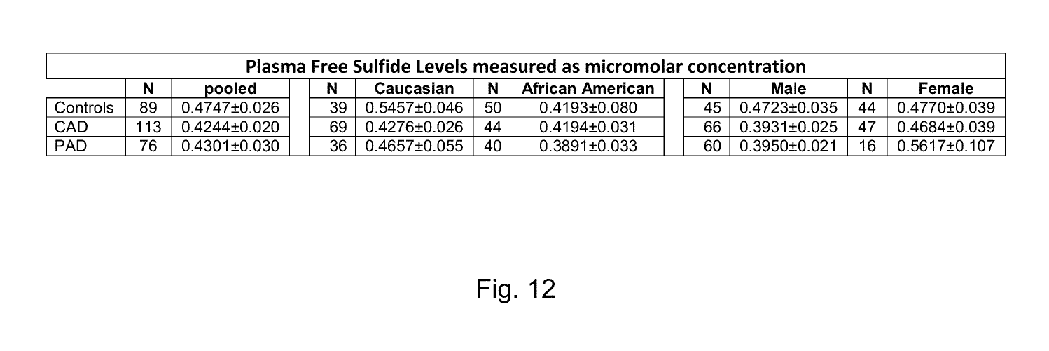

A previous study from our lab showed elevated levels of plasma free H2S in subjects with vascular disease. FIG. 12 shows that free sulfide levels were not significantly different within this current cohort of subjects. An explanation for this observation may be due to the inclusion of subjects with acute coronary syndromes (ACS) or critical limb ischemia (CLI) in our previous study, which are not included here. Importantly, alteration of free H2S is known to occur where hypoxia is an active component of tissue dysfunction and may contribute to our previous findings. Additional studies are planned to examine the relationship between ACS or CLI and plasma sulfide metabolites.

It is well known through numerous studies that African American (AA) subjects are more predisposed to vascular disease. Interestingly, the inventors observed low levels BSS in male AA subjects. Specifically, AA subjects with PAD had reduced BSS compared to controls. Low BSS could potentially be a marker for increased risk for vascular disease in AA subjects. Conversely, lower BSS levels in Caucasian subjects with vascular disease could indicate that BSS has a protective effect in Caucasians that is lost in vascular disease. These observations suggest that BSS may serve as a dynamic sulfide metabolite influencing vascular disease, which requires further study.

An alternative hypothesis is that reduced sulfide metabolites represent deficient endogenous production of sulfide due to the development of CVD itself. Low sulfide levels have been shown to accelerate experimental atherosclerosis, supporting the notion that low BSS is a manifestation of the diseased vasculature. In a study by Mani et al., it was shown that decreased endogenous production of H2S led to accelerated atherosclerosis. In another study, Zavaczki et al. has shown that hydrogen sulfide inhibits the calcification and osteoblastic differentiation of vascular smooth muscle cells and suggested that low H2S could promote vascular calcification seen in atherosclerosis. Several studies demonstrate an increase in production of reactive oxygen species (ROS) in atherosclerosis. BSS is known to be an antioxidant and its low levels could be a manifestation of its utilization and consumption in redox reactions. This may explain the low BSS levels as well as the protective effect of sulfide. That low H2S is a manifestation of the disease state is also supported by the inventors' observation of low BSS under CVD. However, future studies are desired to better understand specific pathophysiological relationships between sulfide bioavailability and vascular disease.

In conjunction with these observations, ethnicity and gender were found to be significant variables associated with total sulfide, ALS, and BSS with a diagnosis of CVD. Regression analyses show a significant reduction in plasma sulfide levels with onset of CVD in Caucasian patients. Notably, a significant association is observed with total sulfide, ALS, and BSS levels in females in comparison to the males in both Caucasians and African Americans. Additionally, SNP analysis of H2S synthesis enzymes revealed that polymorphisms in CTH (but not CBS) are a potential risk factor for vascular disease development. CTH is a key enzyme for production of H2S in the cardiovascular system. Endogenous H2S production is significantly reduced in CTH (CSE) deficient mice, which could translate to clinical implications for the development of vascular disease. Deficiency in bioavailable sulfide may be associated with polymorphic variants of CTH as a previous report revealed an association of CTH SNP with increased serum homocysteine levels. The inventors' findings regarding CTH 1364 G-T allele frequency in patients with CVD advances the hypothesis that a CTH SNP 403Ser to 403Ile is associated with decreased sulfide bioavailability and has clinical associations in patients with chronic vascular disease conditions. However, future studies are desired to identify how this missense mutation alters enzyme function in vivo compared to a previous enzymatic study that did not find alterations in CTH pyridoxal-5′-phosphate cofactor content or steady state kinetic properties.

Study Limitations:

Our study is not without limitations. All subjects were included who presented for cardiac or peripheral arterial catheterization, and for whom an accurate diagnosis could be made. In general, subjects who were scheduled for cardiac catheterization, even if they did not have the clinically defined disease, were usually those with greater cardiovascular risk factors. In an attempt to mitigate this limitation, normal volunteers with less than 2 cardiovascular risk factors and no history of cardiovascular disease were also included in the control group but not subjected to catheterization, as doing this procedure in healthy subjects is not justifiable (for obvious reasons). A second limitation is the fact that ABI was used to detect PAD. Even though ABI is a good screening tool its utility in diagnosing PAD accurately may be limiting, in that a few patients who have PAD could have been missed. Some of our patients had indeterminate ABIs, which would require further clinical workup with an ultrasound modality to rule out PAD if there is high clinical suspicion, which was not done in this study. A third limitation is that the inventors could not assess the role of hypertension as an important risk factor for CVD. This is due to the fact that more than 90% of patients scheduled for cardiac catheterization had hypertension and the inventors were unable to establish a meaningful “normotensive” group. A final limitation was the smaller sample size associated with the genetic polymorphism study. However, this study produced convincing preliminary evidence that mutations in CTH and eNOS genes are higher among patients with CVD, with these initial findings substantiating the need for further investigation. These limitations aside, our findings reveal that reduced sulfide metabolite bioavailability is significantly associated with cardiovascular disease along with increased single nucleotide polymorphisms of CTH. Lastly, additional studies distinguishing specific inorganic and organic per/polysulfides such as cysteine or glutathione-related sulfur species in CVD are desired to better understand the relationship of these metabolites with CVD.

The invention illustratively disclosed herein suitably may explicitly be practiced in the absence of any element which is not specifically disclosed herein. While various embodiments of the present invention have been described in detail, it is apparent that various modifications and alterations of those embodiments will occur to and be readily apparent those skilled in the art. However, it is to be expressly understood that such modifications and alterations are within the scope and spirit of the present invention, as set forth in the appended claims. Further, the invention(s) described herein is capable of other embodiments and of being practiced or of being carried out in various other related ways. In addition, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items while only the terms “consisting of” and “consisting only of” are to be construed in the limitative sense.

Wherefore, I/we claim:

1. A method of diagnosing and treating cardio vascular disease in a patient comprising:
measuring plasma levels of one of free sulfide, acid labile sulfide (ALS), bound sulfane sulfur (BSS), total sulfide metabolites, and some combination thereof;
diagnosing, based on plasma levels, the patient with cardio vascular disease; and
administering a therapeutically effective dose of a pharmaceutical composition to the patient.

2. The method of claim 1 wherein the patient is diagnosed with cardio vascular disease if the patient has a polymorphism in a cystathionine gamma-lyase gene but not a cystathionine-beta-synthase gene.

3. The method of claim 1 wherein the patient is diagnosed with peripheral atrial disease if the patient is African American and the plasma BSS level is significantly lower than an average control plasma BSS level.

4. The method of claim 1 wherein the patient is diagnosed with cardio vascular disease if the patient has a one of lower BSS plasma level and lower total sulfide plasma level than a respective average control BSS plasma level and total sulfide plasma level.

5. The method of claim 1 wherein the patient is diagnosed with cardio vascular disease if the patient has a both lower BSS plasma level and lower total sulfide plasma level than a respective average control BSS plasma level and total sulfide plasma level.

6. The method of claim 1 wherein the patient is diagnosed with cardiac arterial disease and peripheral arterial disease if the patient possesses a CTH 1364 G>T Single Nucleotide Polymorphism.

7. The method of claim 1 wherein the patient is diagnosed with cardiac arterial disease if the patient possesses an eNOS 894 G>T Single Nucleotide Polymorphism.

8. The method of claim 1 wherein the patient is diagnosed with cardiac arterial disease if the patient possesses an eNOS 894 G>T Single Nucleotide Polymorphism and a CTH 1364 G>T Single Nucleotide Polymorphism.

9. The method of claim 1 wherein the patient is diagnosed with one of cardiac arterial disease and peripheral arterial disease if the patient is Caucasian and the plasma total sulfide level is lower than an average plasma total sulfide level.

10. The method of claim 1 wherein the patient is diagnosed with one of cardiac arterial disease and peripheral arterial disease if the patient is Caucasian and the plasma BSS level lower than an average control plasma BSS level.

11. The method of claim 1 wherein the patient is diagnosed with cardiac arterial disease if the patient is an African American and the plasma BSS level is significantly elevated compared to an average plasma BSS level of peripheral arterial disease patients.

12. The method of claim 1 wherein the patient is diagnosed with cardio vascular disease if the patient is a Caucasian female and one of plasma total sulfide level and plasma ALS level is significantly lower than a respective average total sulfide level and plasma ALS level of African American female CVD patients.

13. The method of claim 1 wherein the patient is diagnosed with cardio vascular disease if the patient is a Caucasian female and both plasma total sulfide level and plasma ALS level is significantly lower than a respective average total sulfide level and average plasma ALS level of African American female CVD patients.

14. The method of claim 1 wherein the patient is diagnosed with cardio vascular disease if the patient is a female and one of plasma total sulfide level and plasma ALS level is significantly lower than a respective average control plasma total sulfide level and plasma ALS level.

15. The method of claim 1 wherein the patient is diagnosed with cardio vascular disease if the patient is a female and both plasma total sulfide level and plasma ALS level is significantly lower than a respective average control plasma total sulfide level and plasma ALS level.

16. The method of claim 1 wherein the patient is diagnosed with cardio vascular disease if the patient is Caucasian and one of plasma total sulfide level, plasma ALS level, and plasma BSS level is significantly lower than a respective average control plasma total sulfide level, plasma ALS level, and plasma BSS level.

17. The method of claim 1 wherein the patient is diagnosed with cardio vascular disease if the patient is Caucasian and each of plasma total sulfide level, plasma ALS level, and plasma BSS level is significantly lower than a respective average control plasma total sulfide level, plasma ALS level, and plasma BSS level.

18. The method of claim 1 wherein the patient is diagnosed with cardio vascular disease if the patient is African Americans and has one of lower plasma total sulfide level and lower plasma BSS level compared to respective average African American control plasma total sulfide level and plasma BSS level.

19. The method of claim 1 wherein the patient is diagnosed with cardio vascular disease if the patient is African Americans and has both lower plasma total sulfide level and lower plasma BSS level compared to respective average African American control plasma total sulfide level and plasma BSS level.

20. The method of claim 1 wherein the pharmaceutical composition is one of aspirin, a beta blocker, nitroglycerin, an angiotensin-converting enzyme (ACE) inhibitor, an angiotensin II receptor blocker (ARBs), nitrite, a hypertension medication, a medication to control blood sugar, clopidogrel, cilostazol, pentoxifylline, and a cholesterol-modifying medication, a CTH polymorphism therapeutic, an eNOS polymorphism therapeutic, and some combination thereof.
